(19) United States
(12) Patent Application Publication
(10) Pub. No.: US 2026/0232237 A1
(43) Pub. Date: Aug. 13, 2026

(54) Bidirectional Stopper Valve That Mates to a Venting Syringe

(71) Applicant: Becton, Dickinson and Company, Franklin Lakes, NJ (US)

(72) Inventor: Anthony V. Torris, Mercer Island, WA (US)

(21) Appl. No.: 19/156,438

(22) PCT Filed: Mar. 22, 2024
(86) PCT No.: PCT/US2024/021068

§ 371 Date: Aug. 14, 2025

Related U.S. Application Data
(60) Provisional application No. 63/454,204, filed on Mar. 23, 2023.

(51) Int. Cl. A61B 5/15

(52) U.S. Cl. CPC A61B 5/150992; A61B 5/15003; A61B 5/150213; A61B 5/150221; A61B 5/150351

(57) ABSTRACT

A blood collection system configured for connection to a tube set providing vascular access to a patient, the system including an access device coupled to the tube set, wherein the access device includes a blood collection tube holder and a non-patient needle disposed at least partially within the blood collection tube holder. The system further includes a syringe, wherein the syringe includes a plunger rod, a stopper, and a distal interface, and a cap assembly couplable to the distal interface of the syringe. The cap assembly includes a cap body and a bidirectional valve, and wherein the bidirectional valve is in fluid communication with a venting chamber formed within the cap body.

### CROSS-REFERENCE TO RELATED APPLICATION

The present application claims priority to U.S. Provisional Application Ser. No. 63/454,204 entitled “Bidirectional Stopper Valve that Mates to a Venting Syringe” filed Mar. 23, 2023, the entire disclosure of which is hereby incorporated by reference.

### BACKGROUND OF THE INVENTION

### Field of the Disclosure

The present disclosure relates generally to biological fluid collection devices. More particularly, the present disclosure relates to a cap assembly having a bidirectional stopper valve that mates to a biological fluid collection device such as, e.g., a venting syringe.

### Description of the Related Art

Blood sampling is a common health care procedure involving the withdrawal of at least a drop of blood from a patient. Blood samples are commonly taken from hospitalized, homecare, and emergency room patients either by finger stick, heel stick, or venipuncture. Blood samples may also be taken from patients by venous or arterial lines. Once collected, blood samples may be analyzed to obtain medically useful information including chemical composition, hematology, and coagulation, for example.

For blood draws using existing vascular access, the venous or arterial lines may be coupled to, e.g., a luer lock access device (LLAD) holder or blood transfer device (BTD) holder having a non-patient needle (NPN) provided therein. The holder is configured to receive a blood collection device such as, e.g., a BD VACUTAINER® blood collection tube in order to collect a blood sample from the patient via the venous or arterial lines. The blood collection device is pressed into the holder such that the NPN pierces a rubber stopper of the blood collection tube, with the vacuum in the tube drawing the blood sample into the blood collection device.

However, typical blood collection tubes are generally used to take larger volume blood samples and may require the use of a separate instrument to collect and dispense blood for testing and analysis purposes. Furthermore, typical blood collection tubes are not typically configured for the venting of air bubbles and/or discarding of clotted blood sample prior to delivery for testing or analysis.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In accordance with an aspect of the present disclosure, a blood collection system configured for connection to a tube set providing vascular access to a patient is disclosed, the blood collection system including an access device coupled to the tube set, wherein the access device includes a blood collection tube holder and a non-patient needle disposed at least partially within the blood collection tube holder, and a syringe, wherein the syringe includes a plunger rod, a stopper, and a distal interface. The blood collection system also includes a cap assembly couplable to the distal interface of the syringe, wherein the cap assembly includes a cap body and a bidirectional valve, and wherein the bidirectional valve is in fluid communication with a venting chamber formed within the cap body.

In some embodiments, at least a portion of the bidirectional valve is formed by at least a portion of a stopper member.

In some embodiments, at least a portion of the bidirectional valve is formed by at least a portion of the cap body.

In some embodiments, the stopper member is formed of an elastomeric material piercable by the non-patient needle and self-sealable upon removal of the non-patient needle.

In some embodiments, the cap assembly further includes a top vent member couplable to the cap body, and wherein the top vent member is configured to at least partially retain the stopper member within the cap body.

In some embodiments, the top vent member includes a vent opening in fluid communication with the venting chamber formed within the cap body.

In some embodiments, the vent opening is configured to allow air to escape the venting chamber and prevent a blood sample or other fluid from passing therethrough.

In some embodiments, the cap body further includes a valve opening, the stopper member includes a deflectable portion, and the top vent member includes a notched portion.

In some embodiments, the valve opening, the deflectable portion, and the notched portion act to vent least one of air bubbles and a portion of a blood sample collected within the syringe into the venting chamber formed within the cap body when the plunger rod of the syringe is depressed by a user.

In some embodiments, the distal interface of the syringe is configured as one of a luer lock interface and a luer slip interface.

In some embodiments, the cap body includes a proximal interface coupling configured for removable engagement with the distal interface of the syringe.

In some embodiments, the syringe is configured as an arterial blood gas (ABG) syringe.

In some embodiments, the access device is configured as a luer lock access device.

In some embodiments, exterior contours of at least a portion of the cap body are configured to conform to interior contours of at least a portion of the blood collection tube holder.

According to another aspect of the present disclosure, a cap assembly couplable to a syringe for collection of a blood sample is disclosed. The cap assembly includes a cap body, a stopper member, and a top vent member couplable to the cap body. The top vent member is configured to at least partially retain the stopper member within the cap body, and a venting chamber is formed within the cap assembly between the cap body and the top vent member.

In some embodiments, the cap body includes a valve opening, the stopper member includes a deflectable portion, and the top vent member includes a notched portion.

In some embodiments, the valve opening, the deflectable portion, and the notched portion act to vent least one of air bubbles and a portion of a blood sample collected within a syringe coupled to the cap assembly into the venting chamber when a plunger rod of the syringe is depressed by a user.

In some embodiments, the stopper member is formed of an elastomeric material piercable by a non-patient needle of an access device and self-sealable upon removal of the non-patient needle of the access device.

In some embodiments, the top vent member includes a central sized and configured to accommodate the non-patient needle of the access device such that the non-patient needle is capable of piercing the stopper member.

In some embodiments, the top vent member includes a vent opening in fluid communication with the venting chamber.

In some embodiments, exterior contours of at least a portion of the cap body are configured to conform to interior contours of at least a portion of a blood collection tube holder.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a blood collection system in accordance with an aspect of the present disclosure;

FIG. 2 is a perspective view of a blood collection device and cap assembly in accordance with an aspect of the present disclosure;

FIG. 3 is a partial cross-sectional view of the blood collection system of FIG. 1; and

FIG. 4 is a perspective cross-sectional view of the cap assembly in accordance with an aspect of the present disclosure.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the present disclosure, the distal end of a component or of a device (e.g., a syringe) means the end furthest away from the hand of the user, and the proximal end means the end closest to the hand of the user, when the component or device is in the use position, i.e., when the user is holding a syringe in preparation or during use. Similarly, in this application, the terms “in the distal direction” and “distally” mean in the direction furthest from the hand of the user when in use, and the terms “in the proximal direction” and “proximally” mean in the direction closes to the hand of the user when in use.

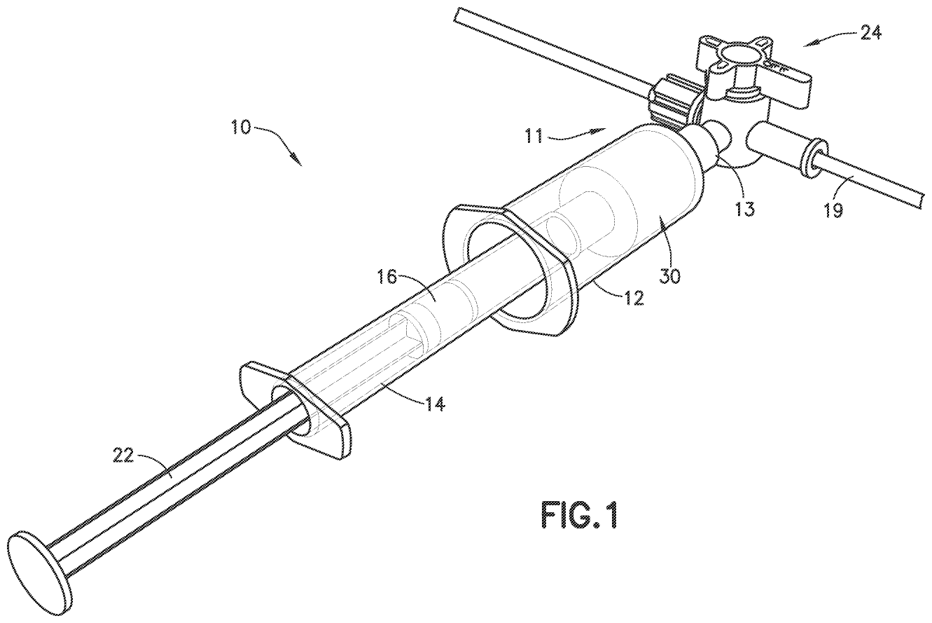

Referring to FIG. 1, a blood collection system 10 in accordance with an aspect of the present disclosure is illustrated. Blood collection system 10 includes a tube set 19 providing vascular access to the patient via, e.g., a catheter. In the embodiment shown in FIG. 1, the tube set 19 is coupled to a three-way valve 24, with one branch of the three-way valve 24 being coupled to an access device 11 such as, e.g., a luer lock access device (LLAD). The access device 11 includes a tube holder 12 and a luer connector interface 13. The three-way valve 24 may be selectively actuated to fluidly couple the access device 11 with the tube set 19 to enable blood collection into an appropriate container. While shown as a luer lock access device (LLAD), it is to be understood that access device 11 may be any appropriate access device such as, e.g., a blood transfer device (BTD). Additionally and/or alternatively, in some embodiments, a three-way valve 24 may not be utilized, with the access device 11 coupled to the tube set 19 via any appropriate interface or coupling.

The holder 12 of access device 11 is sized and configured to receive a blood collection device such as, e.g., a BD VACUTAINER® blood collection tube. However, as discussed above, blood collection tubes such as the BD VACUTAINER® are not typically configured for the venting of air bubbles and/or discarding of clotted blood sample prior to delivery for testing or analysis. Accordingly, the blood collection system 10 in accordance with an embodiment of the present disclosure utilizes a syringe 14 coupled to a cap assembly 30. As will be described in further detail below, the syringe 14 and cap assembly 30 are configured to enable blood collection via the access device 11, while providing for the purging of air and/or clotted blood sample prior to delivery of the sample for testing or analysis.

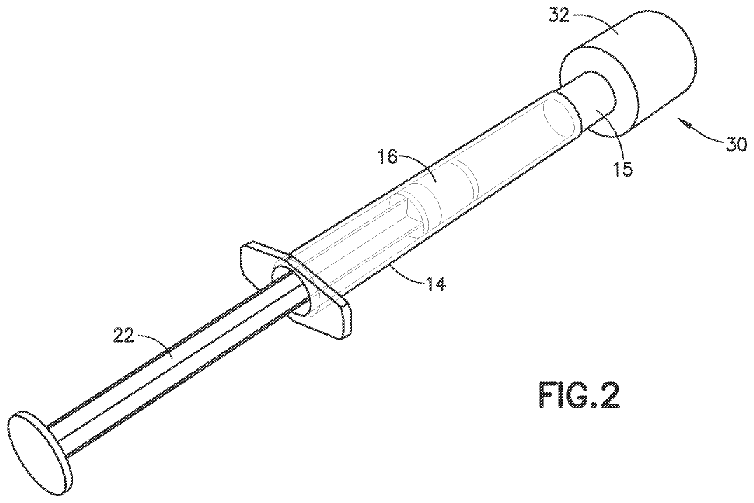

Referring to FIGS. 1 and 2, the syringe 14 includes a stopper 16 and a plunger rod 22. In some embodiments, the syringe 14 may be configured as an arterial blood gas (ABG) vented syringe, which, similar to an evacuated blood collection tube such as the BD VACUTAINER® , may enable blood to be drawn into the syringe 14 via patient blood pressure alone. In other embodiments, the syringe 14 may be configured as a standard syringe such that blood or other fluids are drawn into the syringe 14 via manual proximal displacement of the plunger rod 22 and stopper 16 by the user. The syringe 14 includes a distal interface 15, which may be configured as any appropriate interface such as, e.g., a luer lock interface, a luer slip interface, etc.

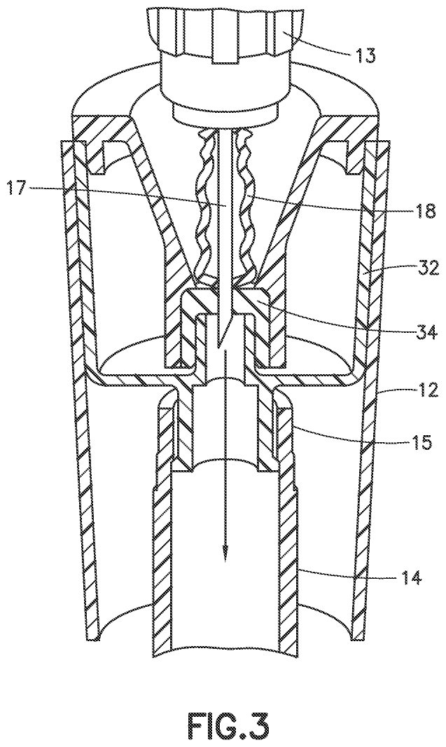

As shown in FIGS. 2 and 3, the cap assembly 30 is configured to be removably coupled to the distal interface 15 of syringe 14. The cap assembly 30 includes a cap body 32. The outer diameter(s) and overall exterior contours of cap body 32 is configured to be substantially similar to that of a cap and stopper component of a conventional blood collection tube used with the access device 11 such as, e.g., the BD VACUTAINER®. In this way, the cap assembly 30 is configured to fit securely (but removably) within a distal end portion of the holder 12, allowing the cap assembly 30 to facilitate blood collection into the syringe 14 via the access device 11, with proper alignment of the cap assembly 30 with a non-patient needle (NPN) 17 of the access device 11.

Specifically, referring to FIG. 3, prior to blood collection via the access device 11, a user inserts the syringe 14 and coupled cap assembly 30 into the holder 12 of the access device 11. Distal movement of the syringe 14 and cap assembly 30 within the holder causes a stopper member 34 of the cap assembly 30 to compress a sheath 18 disposed over the NPN 17, exposing the tip of NPN 17 such that the stopper member 34 is pierced by the NPN 17 to provide a fluid path from the tube set 19 (shown in FIG. 1) into the syringe 14 via the access device 11. The stopper member 34 may be formed of any appropriate material capable of resealing after puncture by the NPN 17 such as, e.g., an elastomeric material, more particularly a thermoplastic elastomer.

After the cap assembly 30 is sufficiently pierced by the NPN 17 as shown in FIG. 3, blood can be collected or drawn into the syringe 14 via any appropriate method. For instance, where syringe 14 is configured as an ABG syringe, blood may be drawn into the syringe 14 via the patient's blood pressure alone. Alternatively, where syringe 14 is a standard syringe, a blood sample may be drawn into the syringe 14 via manual proximal displacement of the plunger rod 22 and stopper 16 by the user. Once the desired blood sample is collected within the syringe 14, the syringe 14 and cap assembly 30 can be withdrawn from the holder 12, thereby removing the NPN 17 from the stopper member 34, which self-seals so as to prevent blood leakage and/or air infiltration into the syringe 14.

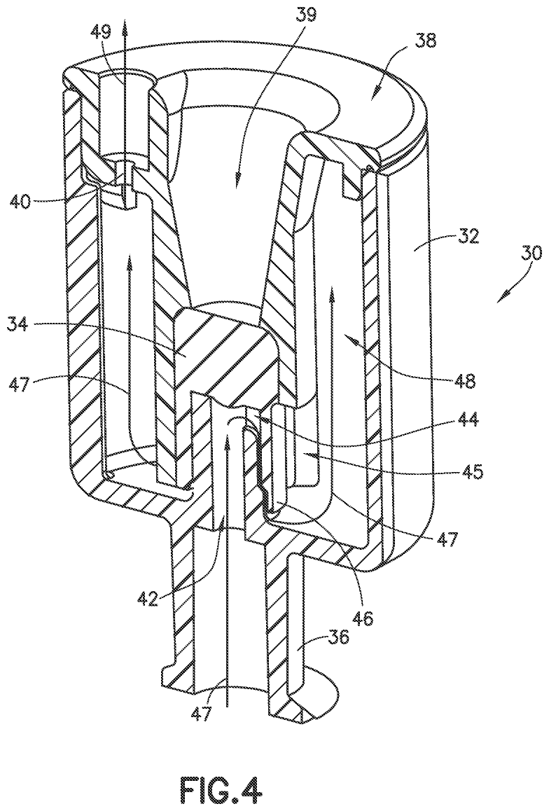

Next, referring to FIG. 4, various features of cap assembly 30 are illustrated. As noted above, the cap assembly 30 includes a cap body 32. A proximal interface coupling 36 extends from the cap body 32 to provide for selective coupling with, e.g., the distal interface 15 of syringe 14. Accordingly, the proximal interface coupling 36 may be configured for threaded engagement with a luer lock interface, press-fit engagement with a luer slip interface, etc.

Furthermore, as discussed above, the cap assembly 30 includes a stopper member 34 formed of, e.g., rubber, and elastomeric material, etc. The stopper member 34 is at least partially retained within the cap assembly 30 by a top vent member 38. The top vent member 38 may be formed of any appropriate material such as, e.g., a polymer or plastic material, and may be formed of the same or a different material than the cap body 32. Additionally, the top vent member 38 may be retained within the cap body 32 by any appropriate method such as, e.g., a press-fit, snap-fit, threaded-fit, etc. Top vent member 38 includes a central cavity 39, with central cavity 39 being sized and configured to accommodate the NPN 17 and sheath 18 of the access device 11 such that the NPN 17 is capable of piercing the stopper member 34 during use.

A channel 42 is formed within the cap body 32 proximally to the stopper member 34. The channel 42 is configured to accommodate at least a tip portion of the NPN 17 during a blood collection procedure, with the channel 42 enabling a blood sample to flow through the proximal interface coupling 36 into the syringe 14. In some embodiments, the coupling between the proximal interface coupling 36 and the distal interface 15 of syringe 14 may allow for automatic mixing of the blood sample as it enters the syringe 14.

Referring still to FIG. 4, the cap body 32 further comprises a valve opening 44 at a position proximal to the stopper member 34. Near the valve opening 44, the top vent member 38 includes a notched portion 45, while the stopper member 34 includes a deflectable portion 46 between the valve opening 44 of the cap body 32 and the notched portion 45 of the top vent member 48.

The interrelationship between the valve opening 44, deflectable portion 46, and notched portion 45 enables the stopper member 34 to operate as a bidirectional stopper valve capable of venting the syringe 14 after a blood collection procedure. That is, after a blood sample is collected into the syringe 14, the syringe 14 and cap assembly 30 can be removed from the holder 12, thereby withdrawing the NPN 17 from the stopper member 34 and resulting in the stopper member 34 self-sealing at the piercing location of NPN 17.

With the syringe 14 and cap assembly 30 removed from the holder 12, the user can purge the syringe 14 of air bubbles and a small volume of clotted blood sample 47 located at or near the luer connector of the syringe 14. To begin this purge, the user typically holds the syringe 14 and cap assembly 30 in a substantially upright position, with the cap assembly 30 disposed vertically above the syringe 14, thereby allowing any air bubbles in the collected sample to rise toward the distal interface 15 of syringe 14. The user then depresses the plunger rod 22 to force air bubbles and/or a portion of the collected blood sample 47 through the channel 42 and into the valve opening 44 of the cap body 32. The air bubbles and/or portion of collected blood sample 47 are forced, by fluid pressure, past the deflectable portion 46 of the stopper member 34, thereby enabling the air bubbles and/or portion of the collected blood sample 47 to enter a venting chamber 48 formed within the cap assembly 30. Purging of any air bubbles and/or clotted sample 47 in this manner provides for improved stabilization of the collected blood sample within the syringe 14 prior to testing and/or analysis.

As shown in FIG. 4, the top vent member 38 may further include a vent opening 40 formed therein, with the vent opening 40 allowing air 49 to pass therethrough during the purge of air bubbles from the collected sample within the syringe 14. While not shown in FIG. 4, it is to be understood that a venting plug or venting filter may be provided at or near the location of the vent opening 40. The venting plug or venting filter may be configured to air to pass therethrough until fully wetted by, e.g., a portion of the blood sample, while preventing a blood sample from passing therethrough. In some embodiments, the venting plug or venting filter include a hydrophobic filter. Thus, the vent opening 40 is effectively blocked when the venting chamber 48 is filled with a portion of the collected blood sample, thereby preventing the leakage of blood sample from the cap assembly 30 via the vent opening 40.

In some embodiments, the cap body 32 and/or the top vent member 38 of the cap assembly 30 may be formed of a substantially transparent or translucent material, thereby enabling the user to visually confirm that a portion of the blood sample is collected in the venting chamber 48.

In addition to providing venting of air bubbles and clotted sample after blood collection, the cap body 32 may also act as a closure for the syringe 14 to prevent blood leaks and/or air infiltration during transport and/or prior to testing and analysis. When testing and analysis of the blood sample within the syringe 14 is desired, the cap body 32 may be removed from the syringe 14 and separately disposed of in an appropriate medical waste container.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A blood collection system configured for connection to a tube set providing vascular access to a patient, the blood collection system comprising:
an access device coupled to the tube set, wherein the access device comprises a blood collection tube holder and a non-patient needle disposed at least partially within the blood collection tube holder;
a syringe, wherein the syringe comprises a plunger rod, a stopper, and a distal interface; and
a cap assembly couplable to the distal interface of the syringe, wherein the cap assembly comprises a cap body and a bidirectional valve, and wherein the bidirectional valve is in fluid communication with a venting chamber formed within the cap body.

2. The blood collection system of claim 1, wherein at least a portion of the bidirectional valve is formed by at least a portion of the cap body.

3. The blood collection system of claim 1, wherein at least a portion of the bidirectional valve is formed by at least a portion of a stopper member.

4. The blood collection system of claim 3, wherein the stopper member is formed of an elastomeric material piercable by the non-patient needle and self-sealable upon removal of the non-patient needle.

5. The blood collection system of claim 3, wherein the cap assembly further comprises a top vent member couplable to the cap body, and wherein the top vent member is configured to at least partially retain the stopper member within the cap body.

6. The blood collection system of claim 5, wherein the top vent member comprises a vent opening in fluid communication with the venting chamber formed within the cap body.

7. The blood collection system of claim 6, wherein the vent opening is configured to allow air to escape the venting chamber and prevent a blood sample or other fluid from passing therethrough.

8. The blood collection system of claim 5, wherein the cap body further comprises a valve opening, the stopper member comprises a deflectable portion, and the top vent member comprises a notched portion.

9. The blood collection system of claim 8, wherein the valve opening, the deflectable portion, and the notched portion act to vent least one of air bubbles and a portion of a blood sample collected within the syringe into the venting chamber formed within the cap body when the plunger rod of the syringe is depressed by a user.

10. The blood collection system of claim 1, wherein the distal interface of the syringe is configured as one of a luer lock interface and a luer slip interface.

11. The blood collection system of claim 10, wherein the cap body comprises a proximal interface coupling configured for removable engagement with the distal interface of the syringe.

12. The blood collection system of claim 1, wherein the syringe is configured as an arterial blood gas (ABG) syringe.

13. The blood collection system of claim 1, wherein the access device is configured as a luer lock access device.

14. The blood collection system of claim 1, wherein exterior contours of at least a portion of the cap body are configured to conform to interior contours of at least a portion of the blood collection tube holder.

15. A cap assembly couplable to a syringe for collection of a blood sample, the cap assembly comprising:
a cap body;
a stopper member; and
a top vent member couplable to the cap body,
wherein the top vent member is configured to at least partially retain the stopper member within the cap body, and
wherein a venting chamber is formed within the cap assembly between the cap body and the top vent member.

16. The cap assembly of claim 15, wherein the cap body comprises a valve opening, the stopper member comprises a deflectable portion, and the top vent member comprises a notched portion.

17. The cap assembly of claim 16, wherein the valve opening, the deflectable portion, and the notched portion act to vent least one of air bubbles and a portion of a blood sample collected within a syringe coupled to the cap assembly into the venting chamber when a plunger rod of the syringe is depressed by a user.

18. The cap assembly of claim 15, wherein the stopper member is formed of an elastomeric material piercable by a non-patient needle of an access device and self-sealable upon removal of the non-patient needle of the access device.

19. The cap assembly of claim 18, wherein the top vent member comprises a central sized and configured to accommodate the non-patient needle of the access device such that the non-patient needle is capable of piercing the stopper member.

20. The cap assembly of claim 15, wherein the top vent member comprises a vent opening in fluid communication with the venting chamber.
